(19) United States
(12) Patent Application Publication
(10) Pub. No.: US 2009/0137840 A1
(43) Pub. Date: May 28, 2009

(54) Fluorinated Alkanesulfonic Acid Anhydrides and Processes for Making the Same

(75) Inventors: Christopher P. Junk, Wilmington, DE (US); Vsevolod Rostovtsev, Swarthmore, PA (US); Allen Capron Sievert, Elkton, MD (US); Lee G. Sprague, Augusta, GA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. Du Pont de Nemours and Company, Wilmington, DE (US)

(21) Appl. No.: 12/274,669

(22) Filed: Nov. 20, 2008

Related U.S. Application Data
(60) Provisional application No. 60/990,730, filed on Nov. 28, 2007.

(51) Int. Cl. C07C 309/06

(52) U.S. Cl. 562/113

(57) ABSTRACT

The invention provides a process for the preparation of fluorinated alkanesulfonic acid anhydrides by contacting alkanesulfonic acids with phosphorus pentoxide, the phosphorus pentoxide being provided as a dispersion in an inert oil. The invention also provides novel fluorinated alkanesulfonic acid anhydrides including 2-hydrotetrafluoroethanesulfonic acid anhydride.

### BACKGROUND OF THE INVENTION

The present invention relates to fluorinated alkanesulfonic acid anhydrides and their preparation.

Because the perfluoroalkanesulfonate anion is an effective leaving group in substitution reactions, perfluoroalkanesulfonic acid esters are used in the alkylation of amines and heterocycles among other applications (see Stang, Hanack, and Subramanian, Synthesis, 1982, issue 2, pp. 85-126). The esters can be made in several ways, but it is convenient to make them by reaction of alcohols with perfluoroalkanesulfonic acid anhydrides.

It is known in the art that perfluorinated sulfonic acid anhydrides can be prepared by contacting the corresponding perfluorinated sulfonic acid with phosphorus pentoxide (P₂O₅), optionally co-mixed with an equal volume of inert material such as sand or diatomaceous earth. The anhydride is isolated by distillation. The use of sand gives low yields and intractable mixtures, and is not amenable to scale-up. Another method for the synthesis of perfluorinated sulfonic acid anhydrides is disclosed by Nakamura, et al. in Japanese Patent No. 3,169,171 B2 and U.S. Pat. No. 5,808,149 wherein a perfluorinated sulfonic acid is contacted with P₂O₅ in a fluorinated solvent such as perfluoro(tripropylamine) or perfluorononane. Due to their low boiling points, common fluorinated solvents are appropriate reaction media only for very volatile anhydrides such as triflic anhydride.

Commonly available perfluoroalkanesulfonic acids are trifluoromethanesulfonic acid (CF₃SO₂OH, triflic acid) and nonafluoro-n-butanesulfonic acid (C₄F₉SO₂OH, nonaflic acid). Lately however, there is growing interest in finding alkanesulfonic acids that are not perfluorinated, but whose esters are still effective alkylating agents. Partially fluorinated alkanesulfonic acids satisfy this criterion.

Thus, there is a need for partially fluorinated alkanesulfonic acid anhydrides as well as convenient methods for their preparation.

### SUMMARY OF THE INVENTION

This invention provides a process for the preparation of a fluorinated alkanesulfonic acid anhydride by reacting a fluorinated alkanesulfonic acid with phosphorus pentoxide, the phosphorus pentoxide being provided as a dispersion in an inert oil. In accordance with a preferred form of the invention, a process is provided for the preparation of a fluorinated alkanesulfonic acid anhydride comprising, (a) reacting a fluorinated alkanesulfonic acid of the formula

(CₛHₜFᵤO)ᵣCₘHₙClₚF_{q}SO₂OH

where m is an integer from 1 to 6, n is an integer from 0 to 2m, p is an integer from 0 to 2, q is an integer from 1 to 2m+1, r is 0 or 1, s is an integer from 1 to 5, t and u are integers from 0 to 2s+1, provided that n+p+q+r=2m+1 and t+u=2s+1 and also provided that m+s<7, with P₂O₅ dispersed in an inert oil, and (b) recovering a fluorinated alkanesulfonic acid anhydride of the formula [(CₛHₜFᵤO)ᵣCₘHₙClₚF_{q}SO₂]₂O.

In one embodiment, this invention provides a process for the preparation of fluorinated alkanesulfonic acid anhydrides of the formula

(R^{f}CHFCF₂SO₂)₂O

where R^{f} is selected from the group consisting of Cl, F, a C₁ to C₄ perfluoroalkyl group, or a C₁ to C₄ perfluoroalkoxy group, comprising, (a) reacting a fluorinated alkanesulfonic acid of the formula R^{f}CHFCF₂SO₂OH with P₂O₅ dispersed in an inert oil, and (b) recovering the fluorinated alkanesulfonic acid anhydride.

In another embodiment, this invention provides a process for the preparation of 2-hydrotetrafluoroethanesulfonic acid anhydride (I)

(CHF₂CF₂SO₂)₂O (I)

comprising, (a) reacting 2-hydrotetrafluoroethanesulfonic acid with P₂O₅ dispersed in an inert oil, and (b) recovering the 2-hydrotetrafluoroethanesulfonic acid anhydride.

The present invention also provides novel fluorinated alkanesulfonic acid anhydrides including those of the formula (R^{f}CHFCF₂SO₂)₂O as defined above. Preferred novel fluorinated alkanesulfonic acid anhydrides include 2-hydrotetrafluoroethanesulfonic acid anhydride (I), 2-hydro-2-chlorotrifluoroethanesulfonic acid anhydride ((CHClFCF₂SO₂)₂O), 2-hydrohexafluoropropanesulfonic acid anhydride ((CF₃CHFCF₂SO₂)₂O), 1,1,2-trifluoro-2-(trifluoromethoxy)ethanesulfonic acid anhydride ((CF₃OCHFCF₂SO₂)₂O), 1,1,2-trifluoro-2-(pentafluoroethoxy)ethanesulfonic acid anhydride ((C₂F₅OCHFCF₂SO₂)₂O), 1,1,2-trifluoro-2-(1,1,1,2,2,3,3-heptafluoroethoxy)ethanesulfonic acid anhydride ((C₂F₅CF₂OCHFCF₂SO₂)₂O), chlorofluoromethanesulfonic acid anhydride ((CHClFSO₂)₂O), difluoromethanesulfonic acid anhydride ((CHF₂SO₂)₂O), 2,2,2-trifluoroethanesulfonic acid anhydride ((CF₃CH₂SO₂)₂O), 1,1,1,2,3,3,3-heptafluoro-2-propanesulfonic acid anhydride ([(CF₃)₂CFSO₂]₂O), 2,2,3,3-tetrafluoropropanesulfonic acid anhydride ((CHF₂CF₂CH₂SO₂)₂O), and 1,1,2,2-tetrafluoro-2-(2,2,2-trifluoroethoxy)ethanesulfonic acid anhydride ((CF₃CH₂OCF₂CF₂SO₂)₂O).

### DETAILED DESCRIPTION OF THE INVENTION

The present invention relates to a process for the preparation of a fluorinated alkanesulfonic acid anhydride by contacting a fluorinated alkanesulfonic acid with phosphorus pentoxide (P₂O₅), the contacting being advantageously carried out employing the phosphorus pentoxide dispersed in an inert oil. As used in this application, the terms “fluorinated alkanesulfonic acid” and “fluorinated alkanesulfonic acid anhydride” means an alkanesulfonic acid and alkanesulfonic acid anhydride, respectively, having at least one fluorine atom, preferably on the carbon atom adjacent to the sulfur atom. The alkane may be straight-chain, branched or cyclic and it may contain one or more ether oxygens and may be substituted with one or more aryl groups. Preferably, fluorine atoms represent at least about 20% of the monovalent atoms of the alkanesulfonic acid and akanesulfonic acid anhydride, more preferably at least about 40%, still more preferably at least about 60%, and most preferably at least about 75%, of the monovalent atoms of the alkanesulfonic acid and alkane sulfonic acid anhydride.

In accordance with a preferred form of the invention, a process is provided for the preparation of a fluorinated alkanesulfonic acid anhydride comprising, (a) reacting a fluorinated alkanesulfonic acid of the formula

(CₛHₜFᵤO)ᵣCₘHₙClₚF_{q}SO₂OH

where m is an integer from 1 to 6, n is an integer from 0 to 2m, p is an integer from 0 to 2, q is an integer from 1 to 2m+1, r is 0 or 1, s is an integer from 1 to 5, t and u are integers from 0 to 2s+1, provided that n+p+q+r=2m+1 and t+u=2s+1 and also provided that m+s<7, with P₂O₅ dispersed in an inert oil, and (b) recovering a fluorinated alkanesulfonic acid anhydride of the formula [(CₛHₜFᵤO)ᵣCₘHₙClₚF_{q}SO₂]₂O.

In one embodiment, this invention provides a process for the preparation of fluorinated alkanesulfonic acid anhydrides of the formula

(R^{f}CHFCF₂SO₂)₂O

where R^{f} is selected from the group consisting of Cl, F, a C₁ to C₄ perfluoroalkyl group, or a C₁ to C₄ perfluoroalkoxy group, comprising, (a) reacting a fluorinated alkanesulfonic acid of the formula R^{f}CHFCF₂SO₂OH with P₂O₅ dispersed in an inert oil, and (b) recovering the fluorinated alkanesulfonic acid anhydride. Represented R^{f} groups include Cl, F, CF₃, OCF₃, OC₂F₅, and OCF₂C₂F₅.

In another embodiment of the present invention, a process is provided for the preparation of 2-hydrotetrafluoroethanesulfonic acid anhydride comprising, (a) reacting 2-hydrotetrafluoroethanesulfonic acid with P₂O₅ dispersed in an inert oil, and (b) recovering the 2-hydrotetrafluoroethanesulfonic acid anhydride.

Examples of fluorinated alkanesulfonic acid anhydrides that may be produced by the process of this invention include (CHClFSO₂)₂O, (CHF₂SO₂)₂O, (CF₃SO₂)₂O, (CF₃CHFSO₂)₂O, (CHF₂CF₂SO₂)₂O, (CHClFCF₂SO₂)₂O, (CF₃CH₂SO₂)₂O, (CF₃CF₂SO₂)₂O, (CF₃CClFSO₂)₂O, (CF₃CF₂CF₂SO₂)₂O, [(CF₃)₂CFSO₂]₂O, (CF₃CHFCF₂SO₂)₂O, (CHF₂CF₂CF₂SO₂)₂O, (CF₃CF₂CHFSO₂)₂O, (CHF₂CF₂CH₂SO₂)₂O, (CF₃OCHFCF₂SO₂)₂O, (C₂F₅OCHFCF₂SO₂)₂O, (C₂F₅CF₂OCHFCF₂SO₂)₂O, (CF₃CH₂OCF₂CF₂SO₂)₂O, (CF₃CF₂CH₂CH₂SO₂)₂O, (CF₃CHFOCF₂CF₂SO₂)₂O, (CHF₂CF₂OCF₂CF₂SO₂)₂O, and (C₄F₉SO₂)₂O.

The fluorinated alkanesulfonic acids used as starting materials in step (a) of the process may be prepared by methods known in the art. For example, the starting material for anhydride (I), 2-hydrotetrafluoroethanesulfonic acid (II, TFESA),

CHF₂CF₂SO₂OH (II)

may be prepared according to the process disclosed in U.S. Patent Application Publication No. 2006/0276671. In this process tetrafluoroethylene (TFE) is reacted with an aqueous solution of potassium sulfite. The reaction product, potassium 2-hydrotetrafluoroethanesulfonate, is then collected, dried, treated with oleum, and the TFESA product is recovered by distillation. Similarly, fluorinated alkanesulfonic acids of the formula R^{f}CHFCF₂SO₂OH where R^{f} is Cl, CF₃, OCF₃, OC₂F₅, and OCF₂C₂F₅, that is (CHClFCF₂SO₂)₂O, CF₃CHFCF₂SO₂OH, CF₃OCHFCF₂SO₂OH, (C₂F₅OCHFCF₂SO₂)₂O, and (C₂F₅CF₂OCHFCF₂SO₂)₂O, may be prepared by the reaction of sodium or potassium sulfite with chlorotrifluoroethylene, hexafluoropropylene, perfluoro(methyl vinyl ether), perfluoro(ethyl vinyl ether), and perfluoro(propyl vinyl ether), respectively, followed by acidification.

1-Hydrotetrafluoroethanesulfonic acid is made through the sultone derived from TFE and sulfur trioxide as reported by Ragulin, et al. in Izvestiya Akademii Nauk SSR, Seriya Khimicheskaya, No. 7, pp. 1560-1564, July 1968. Difluoromethanesulfonic acid may be prepared by reaction of CHClF₂ with sodium sulfite followed by acidification as disclosed by Langlois in French Patent No. 2,504,923. The fluorinated sulfonic acids CF₃CH₂SO₂OH and CHF₂CF₂CH₂SO₂OH may be prepared by conversion of the tosylates of CF₃CH₂OH and CHF₂CF₂CH₂OH, respectively, to the thioethers followed by oxidation and hydrolysis as reported by Bunyagidj, et al. in the Journal of Chemical and Engineering Data, Volume 26, pages 344-346 (1981). Heptafluoro-2-propanesulfonic acid may be prepared by reaction of (CH₃)₂NSF₃ with hexafluoropropene in the presence of cesium fluoride followed by hydrolysis, acidification, and oxidation as reported by Radchenko, et al. in Zhurnal Organicheskoi Khimii, Volume 14, pages 275-278 (1978). 1-Chlorotetrafluoroethanesulfonic acid may be prepared by reaction of (morpholino)₂NSF₃ with chlorotrifluoroethene in the presence of cesium fluoride followed by hydrolysis, acidification, and oxidation as reported by Radchenko, et al. in Zhurnal Organicheskoi Khimii, Volume 16, pages 863-867 (1980). Perfluorinated sulfonic acids such as CF₃SO₂OH and C₄F₉SO₂OH are commercially available. Pentafluoroethanesulfonic acid may be prepared by lithiation of C₂F₅I followed by reaction with SO₂, oxidation of the sulfinate, and acidification as reported by Olah, et al. in Synthesis, 1989, issue 3, pages 463-464. CF₃CH₂OCF₂CF₂SO₂OH may be prepared by reaction of CF₃CH₂OH with the sultone derived from TFE and sulfur trioxide followed by reaction of the resulting ester with SF₄ and hydrolysis of the sulfonyl fluoride intermediate as reported by Cen, et al. in Inorganic Chemistry, Volume 27, pages 1376-1377 (1988). Chlorofluoromethanesulfonic was prepared by catalytic oxychlorofluorination of dimethylsulfide as disclosed by Sweeney, et al. in Canadian Patent Application 1,093,581.

In step (a) of the process of the invention, a fluorinated alkanesulfonic acid is contacted with P₂O₅, the P₂O₅ being provided as a dispersion in an inert oil. By inert oil is meant a fluid that is unreactive with P₂O₅, fluorinated alkanesulfonic acid, and fluorinated alkanesulfonic acid anhydride. Additional characteristics are described below. The dispersions can be prepared by methods known to those skilled in the art. One particularly useful method of preparing dispersions is to add solid particulates to the inert oil mixed with a high shear mixer such as those commercially available from Hockmeyer Equipment Corporation, Harrison, N.J. Such dispersers are available in a variety of blade configurations such as saw tooth, ring blade and vane blade configurations. This operation is preferentially done prior to addition of the fluorinated alkanesulfonic acid to insure a good dispersion of the P₂O₅ reagent. These dispersions can be further mixed by applying higher energy dispersion methods such as sonication, homogenization or microfluidization. Contacting may be carried out by adding the fluorinated alkanesulfonic acid to a vigorously stirred mixture of P₂O₅ and the inert oil. Contact times sufficient for forming the fluorinated alkanesulfonic acid anhydride are typically from about 5 minutes to about 12 hours, preferably about 30 minutes to about six hours. Suitable temperatures for contacting the fluorinated alkanesulfonic acid and P₂O₅ in the inert oil are from about 10° C. to about 100° C., preferably from about 20° C. to about 80° C. The contacting may take place at atmospheric or subatmospheric pressure. If the contacting is performed at subatmospheric pressure, the pressure should be sufficient to maintain at least a portion of the fluorinated alkanesulfonic acid in the liquid phase. The molar ratio of P₂O₅ to fluorinated alkanesulfonic acid is typically from about 1:1 to about 5:1, preferably from about 2.5:1 to about 4:1. Large excesses of P₂O₅ are not beneficial and substoichiometric amounts (i.e., molar ratios of P₂O₅ to fluorinated alkanesulfonic acid of less than 1:1, e.g., 0.8:1) will result in incomplete conversion of the fluorinated alkanesulfonic acid.

The contacting of fluorinated alkanesulfonic acid with P₂O₅ and the inert oil is carried out in a well-agitated reaction vessel suitable for containing highly acidic materials under reduced pressure. The vessels may be fabricated from glass, including glass-lined metal reactors, ceramic, or acid-resistant alloys such as Hastelloy™ C. The reaction vessels and supporting equipment such as mixers and product receivers should be substantially moisture-free.

The ratio of the volume of the inert oil dispersant relative to the weight of P₂O₅ reactant is typically from about 0.75:1 to about 5:1, preferably from about 1:1 to about 2.5:1.

Inert oils suitable for the process of the present invention are those which are stable to highly acidic materials such as fluorinated alkanesulfonic acids and phosphoric acid. Such acid-resistant oils include hydrocarbon oils such as mineral oils (e.g., TW fluids (Inland Vacuum Industries, Churchville, N.Y.)), perfluorinated oils such as perfluoro(polyethers) (e.g., low viscosity Krytox® oils (DuPont, Wilmington, Del.) or Fomblin™ oils (Solvay Solexis, Thorofare, N.J.)), or chlorofluorocarbon oils (e.g., Halovac® 100 or 125 (Inland Vacuum Industries). Other oils suitable for this invention are mixtures of diphenyl oxide and biphenyl, di- and trialkyl ethers, and alkylated aromatics commercially available as Dowtherm® (Dow Chemical, Midland, Mich.). Other oils suitable for this invention are polysiloxanes, especially polydimethyl siloxanes of molecular weights greater than 2,000 [fluids with viscosities greater than 50 cSt (50 mm²/s)]. These materials are available from Dow Corning Midland Mich., as DC-200 fluids. Another class of polysiloxanes useful for this invention are mixed methyl- and diphenyl fluids sold commercially as DC-550 and DC-710 by Dow Corning.

Inert oils suitable for the process of the present invention are further characterized by low vapor pressures in order to permit easy separation of the fluorinated alkanesulfonic acid anhydride product from the oil. Preferred oils have vapor pressures at 25° C. of no greater than about 1×10⁻³ torr (133 mPa), more preferably no greater than about 1×10⁻⁴ torr (13 mPa), and most preferably no greater than about 1×10⁻⁵ torr (1.3 mPa). Typically, these oils have boiling points greater than about 260° C., and preferably greater than 300° C. at atmospheric pressure.

The preferred oils for the preparation of fluorinated alkanesulfonic acid anhydrides of the present invention are chlorofluorocarbon oils. The chlorofluorocarbon oil preferably contains at least about 20 wt % chlorine, more preferably at least about 30 wt % chlorine, and most preferably at least about 40 wt % chlorine, and preferably no more than about 80 wt % chlorine, more preferably no more than about 70 wt % chlorine, and most preferably no more than about 60 wt % chlorine. The non-chlorine monovalent substituents are fluorine and hydrogen, preferably at least as many fluorine atoms as hydrogen atoms, more preferably, at least twice as many fluorine atoms as hydrogen atoms, still more preferably at least four times as many fluorine atoms as hydrogen atoms, and most preferably, only fluorine atoms, with no hydrogen in the chlorofluorocarbon oil.

In step (b) of the process of the invention, the fluorinated alkanesulfonic acid anhydride is recovered by distillation. After contacting the reactants for a period of time sufficient to react at least a substantial portion of the fluorinated alkanesulfonic acid (e.g., greater than 90% of the fluorinated alkanesulfonic acid), the pressure in the reactor is adjusted to a value suitable for distillation of the fluorinated alkanesulfonic acid anhydride product. Suitable pressures for step (b) are from about 100 millitorr (1.33 Pa) to about 50 torr (6.665 kPa), preferably from about 1 torr (13.3 Pa) to about 10 torr (133 Pa) at temperatures of from about 80° C. to about 120° C. Times of from about 0.5 to about 20 hours are sufficient for the reaction forming the anhydride to take place. Preferred times are from about 0.5 to about 5 hours. The temperature and pressure suitable for recovery of a particular fluorinated alkanesulfonic acid anhydride will depend on the vapor pressure of each product. However, distillation temperatures higher than about 120° C. are to be avoided because decomposition of the fluorinated alkanesulfonic acid anhydride typically becomes significant above about 120° C.

The fluorinated alkanesulfonic acid anhydride recovered in this manner is typically sufficiently pure for use as a starting material in other reactions such as the preparation of esters and amides. However, the recovered anhydride may be re-distilled if desired.

After the distillation of the anhydride product, the inert oil may be recovered from the reaction mixture for re-use in subsequent preparations. In one embodiment, the mixture remaining in the reactor after step (b) is treated with water, preferably with cooling. The inert oil and resulting aqueous phosphoric acid solution form separate liquid phases. The density of the inert oil will determine whether it is present as the upper or lower phase in the water-treated mixture. The inert oil is then separated (e.g., by decantation). The recovered oil may be washed with additional portions of water and, optionally, with an aqueous solution of a base such as potassium phosphate, sodium hydroxide, or the like, and then dried by heating at temperatures of from about 50° C. to about 120° C. preferably under vacuum at pressures of from about 1 torr (13.3 Pa) to about 100 torr (1.33 kPa).

The fluorinated alkanesulfonic acid anhydrides of the present invention may be used to prepare aryl esters of the corresponding fluorinated alkanesulfonic acids. The aryl esters can be prepared by adding an aromatic alcohol (i.e., a derivative of phenol) of the formula R¹OH to a fluorinated alkanesulfonic acid anhydride of the formula [(CₛHₜFᵤO)ᵣCₘHₙClₚF_{q}SO₂]₂O in the presence of a base to produce an aryl ester of the formula (CₛHₜFᵤO)ᵣCₘHₙClₚF_{q}SO₂O R¹ where m, n, p, q, r, s, t, and u are as defined above. Aromatic alcohols suitable for forming aryl esters are compounds of the formula HOR¹, where R¹ is a C₆ to C₁₄ aryl group and where the aryl group may be further substituted with one or more substituents selected from the group consisting of R², F, Cl, Br, I, NO₂, CN, OR, C(O)R, CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R².

The molar ratio of fluorinated alkanesulfonic acid anhydride to base is typically from about 0.8:1 to about 1:2, preferably about 1:1. Typically, the fluorinated alkanesulfonic acid anhydride is dissolved or suspended in a non-reactive solvent such as dichloromethane, chloroform, chlorobenzene, toluene, benzotrifluoride, or the like, at a temperature of from about −10° C. to about −40° C., preferably from about −20° C. to about −30° C. The mixture of the anhydride in the solvent is then treated with a base. Suitable bases for the process include nitrogen-containing Lewis bases such as nitrogen heterocycles (e.g. pyridine or 1,8-diazabicyclo[5.4.0]undec-7-ene (DBU)) or a tertiary amine (e.g., triethylamine, 1,4-diazabicyclo[2.2.2]octane (DABCO), or 4-dimethylaminopyridine (DMAP)). The aromatic alcohol may be added as a solution (typically in the same solvent used for the fluorinated alkanesulfonic acid anhydride and base) to the mixture of the fluorinated alkanesulfonic acid anhydride and base with stirring. The rate of aromatic alcohol addition is such that the reaction temperature is maintained at from about −30° C. to about −10° C. When addition is complete, stirring is continued for an additional 30 minutes to 6 hours at about −10° C. to about 0° C.

The molar ratio of aromatic alcohol to the fluorinated alkanesulfonic acid anhydride is typically from about 1:1 to about 1:2, preferably about 1:1.5.

The product aryl ester may be isolated from the reaction mixture using techniques well-known in the art. These techniques may include an aqueous work-up and extraction or, in the case of hydrolytically sensitive materials, distillation.

The fluorinated alkanesulfonic acid anhydrides of the present invention may be used to prepare alkyl esters of the corresponding fluorinated alkanesulfonic acids. The alkyl esters can be prepared by adding an aliphatic alcohol of the formula R⁴OH to a fluorinated alkanesulfonic acid anhydride of the formula [(CₛHₜFᵤO)ᵣCₘHₙClₚF_{q}SO₂]₂O in the presence of a base to produce an alkyl ester of the formula (CₛHₜFᵤO)ᵣCₘHₙClₚF_{q}SO₂OR⁴ where m, n, p, q, r, s, t, and u are as defined above. Aliphatic alcohols suitable for forming alkyl esters are compounds of the formula HOR⁴, where R⁴ is a C₁ to C₁₂ linear, branched, or cyclic alkyl group or a C₇ to C₂₀ aryl-substituted alkyl group. The aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² and R³ are as defined above). The alkyl esters may be prepared by the procedures suitable for the aryl esters discussed above.

The fluorinated alkanesulfonic acid anhydrides of the present invention may be used to prepare amides of the corresponding fluorinated alkanesulfonic acids. The amides can be prepared by adding an amine of the formula NHR⁵R⁶ to a fluorinated alkanesulfonic acid anhydride of the formula [(CₛHₜFᵤO)ᵣCₘHₙClₚF_{q}SO₂]₂O in the presence of a base to produce an amide of the formula (CₛHₜFᵤO)ᵣCₘHₙClₚF_{q}SO₂NR⁵R⁶ where m, n, p, q, r, s, t, and u are as defined above. Amines suitable for producing the amides are compounds of the formula NHR⁵R⁶ where R⁵ is a C₁ to C₁₂ linear, branched, or cyclic alkyl group, or a C₇ to C₂₀ aryl-substituted alkyl group, or a C₆ to C₁₄ aryl group and where the aryl groups may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² and R³ are as defined above and where R⁶ is independently selected from the group consisting of H (hydrogen) and R⁵, and where R⁵ and R⁶ together may form a cyclic structure (e.g. morpholine or carbazole).

The fluorinated alkanesulfonic acid amides may be prepared by the procedures suitable for the aryl esters discussed above except the amine itself may serve as the base. If the amine is used as the base, the molar ratio of amine to the fluorinated alkanesulfonic acid anhydride is typically from about 1.5:1 to about 3:1, preferably about 2:1.

The aryl esters described above may be used as reagents for the arylation of amines of the formula NHR⁵R⁶ to produce amines of the formula NR¹R⁵R⁶ where R¹, R⁵, and R⁶ are as defined above. The arylation may be carried out as described by Anderson et al., in Journal of Organic Chemistry, Volume 68 pages 9563-9574 (2003) and Tundel, et al. in the Journal of Organic Chemistry, Volume 71, pages 430-433 (2006). As illustrated in Example 7, such an arylation takes place in the presence of a suitable catalyst prepared by mixing a palladium compound (e.g., Pd₂(dibenzylideneacetone)₃) with a ligand (e.g., tri-t-butyl phosphine).

The alkyl esters described above may be used as reagents for the alkylation of amines of the formula NHR⁵R⁶ to produce amines of the formula NR⁴R⁵R⁶ where R⁴, R⁵, and R⁶ are as defined above. The alkylation may be carried out as described by Stang, et al. in Synthesis 1982, issue 2, pages 85-126.

### EXAMPLES

### Comparative Example

### Preparation of 2-Hydrotetrafluoroethanesulfonic acid anhydride

An oven-dried 500 mL round-bottom flask is charged with 40 g of sand, 82.0 g (57.7 mmol) of phosphorus pentoxide and a magnetic stir bar. The flask is swirled by hand, becoming warm to the touch. A short-path distillation column is attached and the reaction flask is evacuated and filled with nitrogen atmosphere, twice. 2-Hydrotetrafluoroethanesulfonic acid (41.09 g, 23.0 mmol) is added and the flask is evacuated and filled with nitrogen once more. The reaction mixture is warmed in a 65° C. oil bath and begins to turn dark brown. Reaction mixture is kept at 65° C. for three hours, followed by 16 hours at room temperature. Distillation is carried out at 75° C. under vacuum (60 mtorr, 7 Pa) giving 20.44 g of 2-hydrotetrafluoroethanesulfonic acid anhydride (44% yield), as a clear colorless liquid.

NMR Analysis: ¹H NMR (CD₂Cl₂) 6.31 (2H, tt, ²J_{HF}=51.6 Hz, ³J_{HF}=4.3 Hz). ¹⁹F NMR (CD₂Cl₂) −113.4 (4F, m); −135.5 (4F, dt, ²J_{HF}=51.8 Hz, ³J_{FF}=6.2 Hz).

### Example 1

### Preparation of 2-Hydrotetrafluoroethanesulfonic Acid Anhydride in Chlorofluorocarbon Oil

An oven-dried glassware 2L, 3 neck round-bottom flask is equipped with a mechanical stirrer having a ⅜ inch (9.5 mm) thick Teflon® paddle attached to the shaft, a simple distillation apparatus attached to a recirculating chiller set to −5° C., and a nitrogen/vacuum inlet. The atmosphere in the flask is replaced with nitrogen. The flask is charged with 800 mL of Halovac® 100 chlorofluorocarbon oil and internal pressure is lowered to 2 torr (267 Pa). The reaction flask is refilled with nitrogen again and 500 g (3.5 mol) of phosphorus pentoxide is quickly added through an offset glass funnel while chlorofluorocarbon oil is stirred vigorously. The reaction flask is evacuated and refilled again. A 250 mL addition funnel is attached to the reaction flask and charged with 238 g (1.3 mol) of TFESA. TFESA is then added to the reaction mixture with good stirring over a period of 15 minutes. The flask becomes warm to the touch. The reaction is stirred at room temperature for 30 minutes. Internal pressure is lowered to 2.7 torr (360 Pa), the receiving flask of the distillation apparatus is chilled with liquid nitrogen and, after 15 minutes, several milliliters of clear colorless liquid are collected as a foreshot. A new receiver is placed in the apparatus and the pressure is lowered to 2 torr (266 Pa). A heating mantle is used to slowly apply heat to the flask. The temperature of the distillation is gradually increased from 50° C. to 130° C. One fraction, over a three hour period, is collected to give 167 g (73.8% yield) of 2-hydrotetrafluoroethanesulfonic acid anhydride. NMR analysis confirms the identity of the product.

### Example 2

### Preparation of 2-Hydrotetrafluoroethanesulfonic Acid Anhydride in Perfluoropolyether (PFPE) Oil

Example 1 is repeated with the substitution of Krytox® TLF 8996 oil for Halovac® 100. Krytox® TLF 8996 is a low viscosity perfluoropolyether of the general structure F[CF(CF₃)CF₂O]ₙCF₂CF₃ where n is about 5 to 11. 2-Hydrotetrafluoroethanesulfonic acid anhydride is obtained in a yield of 55%, less than the 73.8% yield in Example 2. This shows the superiority of the chlorofluorocarbon oil over PFPE oil in the preparation of 2-hydrotetrafluoroethanesulfonic acid anhydride from TFESA by reaction with P₂O₅.

### Example 3

### Preparation of Butyl-2-hydrotetrafluoroethanesulfonate

An oven-dried three-neck, 100 mL round-bottom flask, under nitrogen atmosphere, is charged with 30 mL of anhydrous dichloromethane and 0.53 mL (6.5 mmol) of anhydrous pyridine. The reaction mixture is cooled with an ethylene glycol/CO₂ (dry ice) bath to −30° C. A solution of 2.25 g (6.5 mmol) 2-hydrotetrafluoroethanesulfonic acid anhydride in 10 mL of anhydrous dichloromethane is prepared in a drybox and then added by syringe to the reaction mixture, keeping the temperature at −20° C. A solution of 0.28 g (3.8 mmol) of n-butanol in 5 mL of anhydrous dichloromethane is added to the cold reaction mixture. The temperature is maintained at −20° C. during the addition. The reaction is stirred cold for 75 min. Low-boiling volatiles are removed under reduced pressure. The desired product is distilled out of the residue to give 1.3 g (84%) of butyl 2-hydrotetrafluoroethane sulfonate as a clear colorless liquid. The identity of the ester is confirmed by proton and fluorine NMR. This example demonstrates the production in good yield of the butyl ester by reaction of butyl alcohol with 2-hydrotetrafluoroethanesulfonic acid anhydride. This is in contrast to the isomeric 1-hydrotetrafluoroethanesulfonic acid anhydride with which it is possible to make only the methyl and ethyl esters, higher alcohols giving olefin.

### Example 4

### Preparation of 4-tert-Butylphenol-2-hydrotetrafluoroethanesulfonate

An oven-dried three-neck, 250 mL round-bottom flask, under nitrogen atmosphere, is charged with 100 mL of anhydrous dichloromethane and 0.53 mL (6.5 mmol) of anhydrous pyridine. The reaction mixture is cooled with an ethylene glycol/CO2 bath to −30° C. A solution of 2.25 g (6.5 mmol) 2-hydrotetrafluoroethanesulfonic acid anhydride in 20 mL of anhydrous dichloromethane is prepared in a drybox and then added by syringe to the reaction mixture keeping temperature at −20° C. A solution of 0.57 g (3.8 mmol) of 4-tert-butylphenol in 40 mL of anhydrous dichloromethane is added to the cold reaction mixture. The temperature is maintained at −20° C. during the addition. Temperature is maintained and stirring is continued for 75 min. By thin layer chromatography (TLC), using 25% ethyl acetate/hexane, 4-tert-butylphenol is found to be absent. Stirring is continued at −10° C. for a further 3 hours, then the reaction mixture is poured onto 200 mL of 5% NaHCO₃. The layers are separated and the organic layer is dried over Na₂SO₄. The solvent is removed and the crude product is purified on a silica gel column (10% ethyl acetate/hexane) to yield 1.0 g (84%) of 4-(tert-butylphenyl)-2-hydrotetrafluoroethane sulfonate as a clear colorless liquid.

NMR Analysis: ¹H NMR (CDCl₃) 1.32 (9H, s); 6.23 (1H, tt, ²J_{HF}=52.3 Hz); 7.19 (2H, app d); 7.43 (2H, app d). ¹⁹F NMR (CDCl₃) −117.15 (2F,m); −135.3 (2F, dt, ²J_{HF}=52.3 Hz).

Elementary Analysis: Calculated: % C 45.86; % H 4.49; % F, 24.18; % S, 10.2. Found: % C 46.24; % H 5.03; % F, 24.44; % S, 9.78.

### Example 5

### Preparation of N,N-dimethyl-2-hydrotetrafluoroethanesulfonamide

Following the general procedure of Example 3, 2-hydrotetrafluoroethanesulfonic acid anhydride is reacted with dimethyl amine in place of n-butanol. The product is substantially all N,N-dimethyl-2-hydrotetrafluoroethanesulfonamide with no detectable amounts of the product of reaction of 2-hydrotetrafluoroethanesulfonic acid anhydride with two molecules of dimethyl amine. This example demonstrates that 2-hydrotetrafluoroethanesulfonic acid anhydride reacts cleanly with amines to make amides without significant side reaction, unlike the isomeric 1-hydrotetrafluoroethanesulfonic acid anhydride in which reaction with two molecules of amine can be the predominant reaction.

### Example 6

### Preparation of N-(4-tert-butylphenyl)aniline

In a drybox, a glass thick-walled pressure tube is charged with 0.055 mL (0.6 mmoL) of aniline, 0.157 g (0.5 mmol) of 4-tert-butylphenyl 2-hydrotetrafluoroethane sulfonate, 0.067 g (0.7 mmol) of sodium t-butoxide, 0.02 g (0.05 mmol) of 2-(dicyclohexylphosphino)-2′-(N,N-dimethylamino)biphenyl, 0.023 g (0.025 mmol) of palladium dibenzylideneacetone and 2 mL of toluene. The tube is sealed, brought out of the drybox and heated at 80° C. for 16 h. The reaction mixture is cooled to room temperature, the tube is opened, and ether is added and the mixture is passed through a small plug of celite. The solvents are removed in vacuo. The crude product is purified on a 20 g silica gel column using 5% ethyl acetate/hexane. Removal of volatiles yields 0.096 g (86%) of N-(4-tert-butylphenyl)aniline as a light brown oil. The proton NMR is identical to that of N-(4-tert-butylphenyl)aniline as given in the literature. This reaction goes in good yield is in contrast to the reaction of 4-tert-butylphenyl 1-hydrotetrafluoroethane sulfonate with aniline, which gives a complex mixture of products.

What is claimed is:

1. A process for the preparation of a fluorinated alkanesulfonic acid anhydride from a fluorinated alkanesulfonic acid comprising contacting said fluorinated alkanesulfonic acid with a dispersion of phosphorus pentoxide in an inert oil and recovering the fluorinated alkanesulfonic acid anhydride.

2. The process of claim 1 wherein the fluorinated alkanesulfonic acid is of the formula
(CₛHₜFᵤO)ᵣCₘHₙClₚF_{q}SO₂OH
where m is an integer from 1 to 6, n is an integer from 0 to 2m, p is an integer from 0 to 2, q is an integer from 1 to 2m+1, r is 0 or 1, s is an integer from 1 to 5, t and u are integers from 0 to 2s+1, provided that n+p+q+r=2m+1 and t+u=2s+1 and also provided that m+s<7.

3. The process of claim 1 wherein the fluorinated alkanesulfonic acid is of the formula
R^{f}CHFCF₂SO₂OH
wherein R^{f} is selected from the group consisting of Cl, F, a C₁ to C₄ perfluoroalkyl group, or a C₁ to C₄ perfluoroalkoxy group.

4. The process of claim 3 wherein the fluorinated alkanesulfonic acid is 2-hydrotetrafluoroethanesulfonic acid.

5. The process of claim 1, wherein the inert oil comprises a chlorofluorocarbon oil.

6. The process of claim 1 wherein said recovering of the fluorinated alkanesulfonic acid anhydride comprises distilling said fluorinated alkanesulfonic acid anhydride from said inert oil.

7. A fluorinated alkanesulfonic acid anhydride of the formula
(R^{f}CH FCF₂SO₂)₂O
where R^{f} is selected from the group consisting of Cl, F, a C₁ to C₄ perfluoroalkyl group, or a C₁ to C₄ perfluoroalkoxy group.

8. The fluorinated alkanesulfonic acid anhydride of claim 7 wherein said acid anhydride is 2-hydrotetrafluoroethanesulfonic acid anhydride.

9. The fluorinated alkanesulfonic acid anhydride of claim 7 wherein said acid anhydride is 2-hydrohexafluoropropanesulfonic acid anhydride.

10. The fluorinated alkanesulfonic acid anhydride of claim 7 wherein said acid anhydride is 1,1,2-trifluoro-2-(trifluoromethoxy)ethanesulfonic acid anhydride.

11. The fluorinated alkanesulfonic acid anhydride of claim 7 wherein said acid anhydride is 1,1,2-trifluoro-2-(pentafluoroethoxy)ethanesulfonic acid anhydride.

12. The fluorinated alkanesulfonic acid anhydride of claim 7 wherein said acid anhydride is 1,1,2-trifluoro-2-(1,1,1,2,2,3,3-heptafluoroethoxy)ethanesulfonic acid anhydride.

13. The fluorinated alkanesulfonic acid anhydride of claim 7 wherein said acid anhydride is 2-hydro-2-chlorotrifluoroethanesulfonic acid anhydride.

14. Difluoromethanesulfonic acid anhydride.

15. 2,2,2-Trifluoroethanesulfonic acid anhydride.

16. 1,1,1,2,3,3,3-Heptafluoro-2-propanesulfonic acid anhydride.

17. 2,2,3,3-Tetrafluoropropanesulfonic acid anhydride.

18. 1,1,2,2-Tetrafluoro-2-(2,2,2-trifluoroethoxy)ethanesulfonic acid anhydride.
